(19) United States
(12) Patent Application Publication
(10) Pub. No.: US 2021/0031455 A1
(43) Pub. Date: Feb. 4, 2021

(54) 3D BIOPRINTING A MEDICAL DEVICE THROUGH FREEFORM REVERSIBLE EMBEDDING

(71) Applicant: Carnegie Mellon University, Pittsburgh, PA (US)

(72) Inventors: Adam FEINBERG, Pittsburgh, PA (US); Andrew HUDSON, Pittsburgh, PA (US)

(73) Assignee: Carnegie Mellon University, Pittsburgh, PA (US)

(21) Appl. No.: 17/045,907

(22) PCT Filed: Apr. 10, 2019
(86) PCT No.: PCT/US2019/026787

§ 371 Date: Oct. 7, 2020

Related U.S. Application Data
(60) Provisional application No. 62/761,897, filed on Apr. 10, 2018.

(51) Int. Cl. B29C 64/386; A61L 27/22; A61L 27/24; A61L 27/36; A61L 27/20; A61L 27/54; A61L 27/52; B29C 64/40; B33Y 50/00; B33Y 10/00

(52) U.S. Cl. CPC B29C 64/386; A61B 5/0066; A61L 27/24; A61L 27/3633; A61L 27/20; A61L 27/54; A61L 27/3687; A61L 27/52; B29C 64/40; B33Y 50/00; B33Y 10/00; A61L 2430/02; A61L 2430/20; A61L 2430/22; A61L 2430/30; A61L 2430/06; A61L 2430/32; A61L 27/225

(57) ABSTRACT

Various systems and process for fabricating customized medical devices via the freeform reversible embedding of suspended hydrogels process are disclosed. The mechanical properties of the fabricated objects can be controlled according to the manner or orientation in which the structure material is deposited into the support material and the three-dimensional movement of the extruder assembly. Further, the dimensions of the fabricated objects can be validated by adding a contrast agent to the structure material, obtaining a three-dimensional reconstruction of the fabricated object, and then comparing the three-dimensional reconstruction to the computer model upon which the fabricated object is based. These and other techniques are described herein.

### PRIORITY

This application claims priority under 35 U.S.C. § 119(e) to U.S. provisional patent application No. 62/761,897, titled 3D BIOPRINTING A HYDROGEL-BASED MEDICAL DEVICE THROUGH FREEFORM REVERSIBLE EMBEDDING OF SUSPENDED HYDROGELS, filed Apr. 10, 2018, which is hereby incorporated by reference herein in its entirety.

### BACKGROUND

Additive manufacturing (AM) of biological systems has the potential to revolutionize the engineering of soft structures, bioprosthetics, and scaffolds for tissue repair. While three-dimensional (3D) printing of metals, plastics, and ceramics has radically changed many fields, including medical devices, applying these same techniques for the printing of complex and soft biological structures has been limited. The major challenges are (i) deposition of soft materials with elastic moduli of less than 100 kilopascal (kPa), (ii) supporting these soft structures as they are printed so they do not collapse, (iii) removing any support material that is used, and (iv) keeping cells alive during this whole process using aqueous environments that are pH, ionic, temperature, and sterility controlled within tight tolerances. Expensive bioprinters that attempt to address these challenges have been produced but have yet to achieve results using soft hydrogels that are comparable to results achieved using commercial-grade thermoplastic printers.

Some hydrogels are impossible to deposit in layers due to their tendency to flow or deform under steady-state loading. However, hydrogels are desirable materials for advanced biofabrication techniques because their structure underlies the function of complex biological systems, such as human tissue. 3D tissue printing (i.e., AM of tissues) seeks to fabricate macroscopic living composites of biomolecules and cells with relevant anatomical structure, which gives rise to the higher-order functions of nutrient transport, molecular signaling, and other tissue-specific physiology. Replicating the complex structures of tissues with AM requires true freeform fabrication, as tissues possess interpenetrating networks of tubes, membranes, and protein fibers that are difficult to fabricate using free-standing fused-deposition or photopolymerization techniques. Conventional AM techniques may not possess the level of spatial control necessary for freeform fabrication and rapid prototyping of soft tissues.

Recent advances in 3D tissue printing represent solutions to highly specific problems encountered in the AM of hydrogel materials and are often limited to a specific application. For example, Fused Deposition Modeling (FDM) has been used to print avascular replicas of cartilaginous tissues as well as fugitive vasculatures, which can be used to cast a vascularized tissue. Similar to the powders used in Solid Freeform Fabrication, dynamic support materials have been developed to enable the fabrication of soft materials in complex spatial patterns without the need of printed supports. These semi-solid materials may be capable of supporting the fusion of cells and gels; however, the latter cases are limited and do not constitute true freeform fabrication. Indeed, the most successful methods for fabricating macroscopic biological structures in vitro rely on casting and not AM, as conventional AM techniques may not be sufficient to recreate true tissue complexity.

Many gels are ideal materials for biofabrication, because their structures underlie the function of complex biological systems, such as human tissues. The geometries of tissues may be difficult to recreate without techniques like AM/3D printing, but the methods for 3D printing gels are limited. Many gels start as fluids and cannot be 3D printed without supports to prevent them from drooping or oozing. Conventional 3D printing techniques may not possess the level of control necessary for geometrically unrestrained 3D printing of gels and tissues. Attempts to print gels with FDM have yielded cartilage-like tissues as well as gels with simple networks of vessels, yet the results have been limited. Indeed, it is still easier and more effective to cast a tissue than it is to 3D print it, as conventional 3D printing techniques may not be sufficiently capable.

Further, fabricating medical devices, such as replacement biological structures, tissue scaffolds, and nerve guidance conduits, from extracellular matrix (ECM) materials and related materials would provide several benefits. For example, such medical devices would have mechanical, electrical, and/or structural properties commensurate with naturally occurring biological structures. As another example, such medical devices would have improved biointegration characteristics and thus suffer from fewer post-surgical complications due to a lack of biological compatibility between the medical device and the patient. Still further, medical devices could be fabricated from decellularized tissue harvested directly from the patient or the tissue or structure being replaced or segmented by the medical device. In this way, such a medical device could be specifically tailored for each individual patient.

### SUMMARY

The present invention, in one general aspect, is designed to provide additively printed, biocompatible, functional, patient-customized, medical devices, such as replacement biological structures, nerve guidance conduits, and tissue scaffolds.

In another general aspect, the present invention is directed to systems and methods for, in various embodiments, fabricating a medical device, such as a replacement structure for a biological structure of a patient, by depositing a structure material into a support material in the form of the replacement structure based upon a computer model generated from image data of the biological structure of the patient, removing the support material, and inducing cross-linking of the structure material of the replacement structure. The support material is configured to be stationary at an applied stress level below a threshold shear stress level and flows at an applied shear stress level at or above the threshold shear stress level. Further, the support material is configured to physically support the structure material during deposition of the structure material. The structure material comprises a fluid that transitions to a solid or semi-solid state after deposition.

In another general aspect, the present invention is directed to a patient-customized medical device that has been fabricated according to the processes described above.

Embodiments of the present invention can open the possibility of reducing reliance on organ and tissue donors by creating high-quality, high-resolution, individualized, patient-specific medical devices out of soft materials. These and other benefits of the present invention will be apparent from the description that follows.

### FIGURES

The features of various aspects are set forth with particularity in the appended claims. The various aspects, however, both as to organization and methods of operation, together with further objects and advantages thereof, may best be understood by reference to the following description, taken in conjunction with the accompanying drawings as follows.

FIGS. 1A-1D illustrate a structure being fabricated via the Freeform Reversible Embedding of Suspended Hydrogels (FRESH) process, in accordance with at least one aspect of the present disclosure.

FIG. 2 is a graph of compression testing data of an alginate structure fabricated utilizing the FRESH process, in accordance with at least one aspect of the present disclosure.

FIG. 3 is a graphical comparison of compression testing data for various structures fabricated utilizing the FRESH process, in accordance with at least one aspect of the present disclosure.

FIG. 4 is a flow diagram of a process for fabricating customized biological structures, in accordance with at least one aspect of the present disclosure.

FIG. 5 is an image of a heart valve fabricated according to the process of FIG. 4, in accordance with at least one aspect of the present disclosure.

FIGS. 6A and 6B are images of an alginate heart valve closing and opening in response to pulsatile flow, in accordance with at least one aspect of the present disclosure.

FIGS. 6C and 6D are images of a collagen heart valve closing and opening in response to pulsatile flow, in accordance with at least one aspect of the present disclosure.

FIG. 7 is a graph of Doppler flow velocimetry across a collagen valve, in accordance with at least one aspect of the present disclosure.

FIG. 8A is a graphical representation of a heart valve, in accordance with at least one aspect of the present disclosure.

FIG. 8B is the graphical representation of FIG. 8A after being processed by slicing software, in accordance with at least one aspect of the present disclosure.

FIG. 8C is a sectional view of the graphical representation of FIG. 8B with a 50% infill density, in accordance with at least one aspect of the present disclosure.

FIG. 8D is a sectional view of the graphical representation of FIG. 8B with a 10% infill density, in accordance with at least one aspect of the present disclosure.

FIG. 9A is an image of a portion of a collagen heart valve and a higher magnification thereof, in accordance with at least one aspect of the present disclosure.

FIG. 9B is an image of two leaflets of a collagen heart valve and increasing higher magnifications thereof, in accordance with at least one aspect of the present disclosure.

FIG. 10 is a flow diagram of a process for gauging a fabricated structure, in accordance with at least one aspect of the present disclosure.

FIG. 11A is a computerized tomography (CT) scan of an additively manufactured heart valve, in accordance with at least one aspect of the present disclosure.

FIG. 11B is a sectional view of the CT scan in FIG. 11A, in accordance with at least one aspect of the present disclosure.

FIG. 11C is a 3D model of the heart valve shown in FIGS. 11A and 11B, in accordance with at least one aspect of the present disclosure.

FIG. 11D is an overlay of the 3D model shown in FIG. 11C and the image shown in FIG. 11A, in accordance with at least one aspect of the present disclosure.

FIG. 11E is a surface deviation analysis of the overlay shown in FIG. 11D, in accordance with at least one aspect of the present disclosure.

FIG. 12 is a block diagram of an AM system, in accordance with at least one aspect of the present disclosure.

### DESCRIPTION

Certain aspects will now be described to provide an overall understanding of the principles of the structure, function, manufacture, and use of the devices and methods disclosed herein. One or more examples of these aspects are illustrated in the accompanying drawings. Those of ordinary skill in the art will understand that the devices and methods specifically described herein and illustrated in the accompanying drawings are non-limiting example aspects and that the scope of the various aspects is defined solely by the claims. The features illustrated or described in connection with one aspect may be combined with the features of other aspects. Such modifications and variations are intended to be included within the scope of the claims. Furthermore, unless otherwise indicated, the terms and expressions employed herein have been chosen for the purpose of describing the illustrative aspects for the convenience of the reader and are not to limit the scope thereof.

### FRESH Process

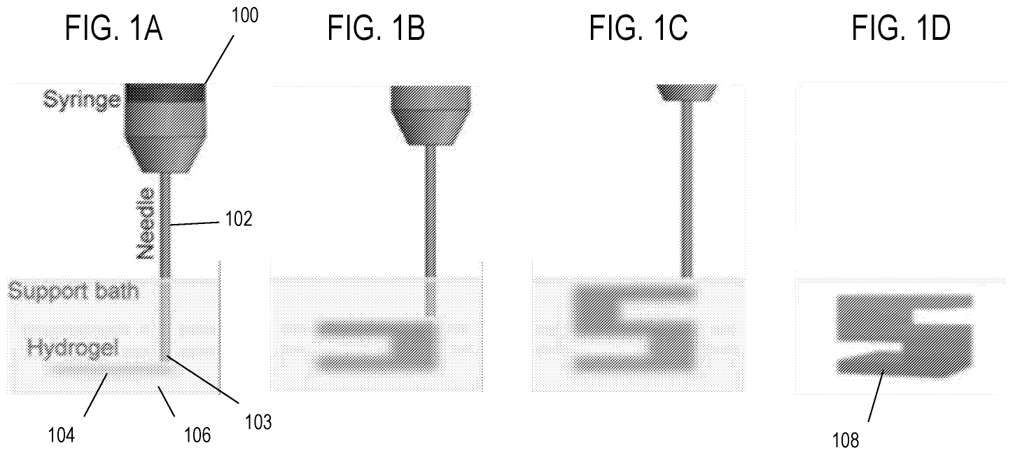

FRESH is an example of a freeform reversible embedding (FRE) technique for fabricating an object. FRE techniques are AM processes by which a structure material is deposited and embedded into a support material (referred to as a “support bath,” in some instances) that physically supports and maintains the intended geometry of the embedded structure material during the manufacturing process. Although the techniques described herein are primarily discussed in terms of the FRESH process, this is merely for illustrative purposes and it should be understood that the techniques are generally applicable to any FRE process. In one implementation of a FRE process, referring to FIG. 1A, the structure material 104 can be deposited via an extruder assembly, which can include a syringe 100 housing the structure material 104 and a needle 102 through which the structure material 104 is extruded. In one aspect, the extruder assembly can further including a gantry supporting the syringe 100, a motor assembly or other movement assembly configured to translate and/or rotate the gantry, the syringe 100 and/or the platform on which the support material 106 rests, and an actuator (e.g., a motor) configured to depress a plunger to extrude the structure material 104 from the syringe 100 through the needle tip 103 into the support material 106 as the needle 102 is translated through the support material 106, as shown in FIGS. 1A-1C, to form a 3D object 108. After deposition of the structural material 104 has been completed, the support material 106 is then removed to release the 3D object 108, as shown in FIG. 1D. As with other AM techniques, the 3D object 108 fabricated by the extruder assembly is based upon a computer model. The computer model is sliced into a series of layers (e.g., by Skeinforge or KISSlicer software), which are then utilized to generate a set of instructions (e.g., G-code instructions) for controlling the movement of the extruder assembly to form the 3D object 108 defined by the computer model from the structure material 104.

In one aspect, the 3D object 108 can be a replacement human body part or biological structure and the structure material 104 can include hydrogels, bioinks, and/or other biomaterials. In the FRESH process, the structure material 104 includes hydrogels. The hydrogels can be formed from ECM materials, such as natural polymers (e.g., collagen), polysaccharides (e.g., alginate or hyaluronic acid), glycoproteins (e.g., fibrinogen), decellularized ECM materials, and ECM-based materials (e.g., Matrigel, which is a mixture of structural proteins such as laminin, nidogen, collagen, and heparan sulfate proteoglycans, secreted by Engelbreth-Holm-Swarm mouse sarcoma cells). In one aspect, the hydrogel structure material 104 can be formed from decellularized ECM materials or tissue harvested from the patient's biological structure being replaced or augmented with the FRE-fabricated object 108. In this way, the properties of the object 108 can be precisely tailored to the properties of the biological structure at issue. In one aspect, the support material 106 can include a Bingham plastic or Bingham plastic-like material. Such materials behave as a rigid body at low shear stresses but flow as a viscous fluid at higher shear stresses. Accordingly, the support material 106 provides little mechanical resistance to the needle 102 as it is translated therethrough, but physically supports and holds in place the deposited structure material 104. Thus, the support material 106 can maintain soft materials (e.g., the structure material 104) that would collapse if they were printed outside of the support material 106 in the intended 3D geometry. As one example, the support material 106 can include a slurry of gelatin microparticles processed to have a Bingham plastic rheology, as described in Hinton et al. (2015), Three-dimensional printing of complex biological structures by freeform reversible embedding of suspended hydrogels, Science Advances 1, e1500758. In one aspect, the support material 106 can be tailored to match the gelation mechanism of the structure material 104, such as exposure to divalent cations (e.g., Ca²⁺) for alginate or pH neutralization for collagen. In one aspect, the support material 106 can comprise a thermoreversible material. Accordingly, the 3D object 108 can be released from the support material 106 by heating the support material 106 from an operational temperature (e.g., 22° C.) at which the 3D object 108 is fabricated to a threshold temperature (e.g., 37° C.) that causes the support material 106 to melt away from the object 108 nondestructively.

In various aspects, the object 108 can be treated through various cross-linking techniques to selectively increase the rigidity of the overall object 108 or portions thereof. In some aspects, the step of inducing cross-linking in the structure material 104 of the object 108 can be skipped. In one aspect, the support material 106 can include a cross-linking agent for treating the structure material 104 as it is deposited into the support material 106. For example, the support material 106 can include divalent cations (e.g., 0.16% CaCl₂) to induce cross-linking in the structure material 104 while it is embedded in the support material 106. In another aspect, the structure material 104 can be treated via a variety of different cross-linking techniques after the object 108 has been released from the support material 106. For example, the released object 108 can be treated with a cross-linking agent or via photo-induced cross-linking techniques (e.g., Photo-Induced Cross-Linking of Unmodified Proteins) to induce cross-linking of the support material 106.

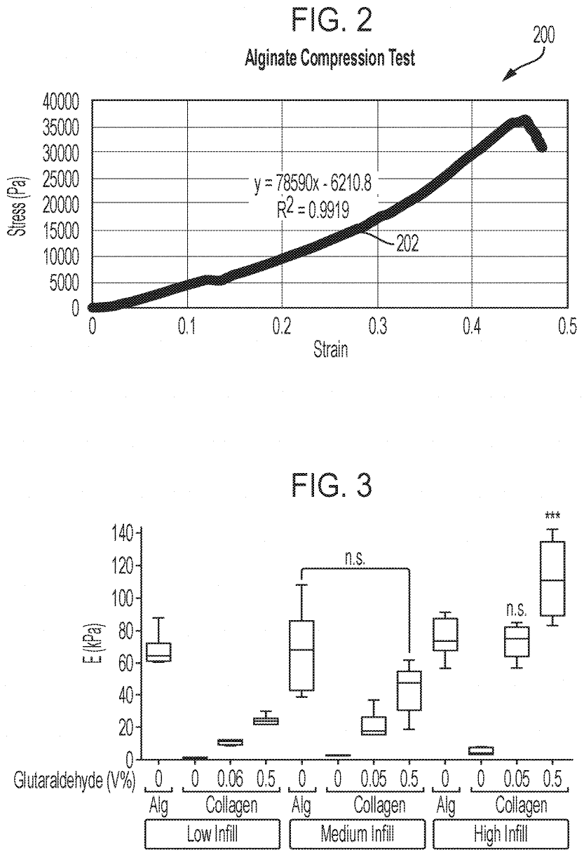

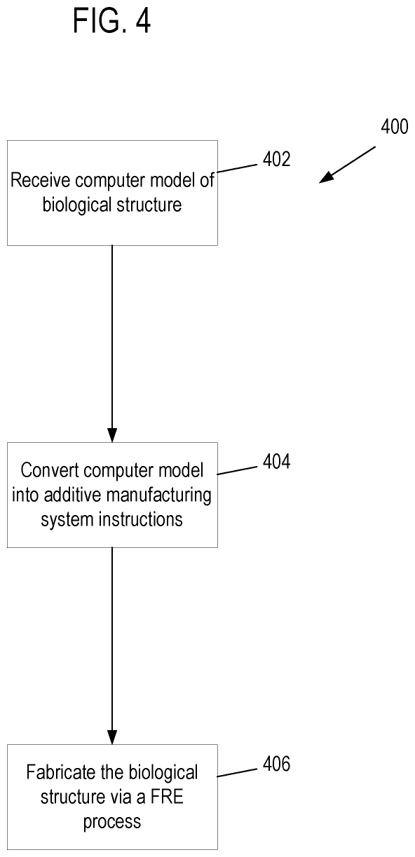

Further, the amount or type of cross-linking can be selected based upon the type of structure material 104 utilized to fabricate the object. For example, collagen may have a lower mechanical strength than alginate. To increase collagen's mechanical strength to match that of alginate, collagen structure material can be, for example, fixed for seven days in various concentrations of glutaraldehyde at 0.05% (v/v) and 0.5% (v/v) along with 1× phosphate buffered saline (PBS) to serve as the control along with standard alginate-fabricated objects fixed in 1% (w/v) CaCl₂. During testing, the mechanical properties of objects fabricated from different structure materials 104 and with different amounts of cross-linking were validated utilizing compression cylinders fabricated from the structure materials 104. Compression cylinders having dimensions of 10 mm×5 mm (D×h) were fabricated using the FRESH process from either 23 mg/ml acidified collagen or 4% (w/v) alginic acid using a nozzle having a diameter of 150 μm. Compression cylinders (n=6 of each type) were printed at 35%, 50%, or a near-solid infill of 75% or 90% for alginate and collagen, respectively, using a 60 μm layer height. The diameter of each cylinder was measured before mechanical testing. Compression testing was performed on an Instron 5943 tensile and compression testing instrument at a strain rate of 1 mm/min until approximately 60% strain. The elastic modulus of each sample was calculated from the slope of the linear elastic region 202 (FIG. 2) of the stress-strain curves. For the particular data set represented by the graph 200 illustrated in FIG. 2, the linear elastic region 202 was determined to extend from 15-35% strain. The compressive moduli of these samples were compared by an analysis of variance (ANOVA) with a Tukey post-hoc comparison (FIG. 3).

During testing, collagen samples were compared to alginate samples of respective infill with the goal of matching collagen's compressive modulus to alginate's at a similar infill. At low infill, test samples fabricated from collagen were experimentally shown to be weaker than test samples fabricated from alginate. At medium infill, test samples fabricated from collagen fixed with 0.5% (v/v) glutaraldehyde were statistically similar to test samples fabricated from alginate. At high infill, test samples fabricated from collagen fixed with 0.05% (v/v) glutaraldehyde were statistically similar to test samples fabricated from alginate, whereas 0.5% (v/v) glutaraldehyde fixation results in test samples fabricated from collagen being stiffer than test samples fabricated from alginate.

In sum, the FRESH process (and other FRE processes) generally includes the steps of: (i) depositing a structure material into a support material according to a computer model of the structure to be fabricated, where the support material is configured to physically support and maintain the structure material in the intended 3D shape; (ii) removing the support material; and optionally (iii) cross-linking the structure material of the fabricated object either prior to or after the support material has been removed. Additional details regarding the FRESH process can be found in U.S. Pat. No. 10,150,258, titled ADDITIVE MANUFACTURING OF EMBEDDED MATERIALS, filed Jan. 29, 2016, which is hereby incorporated by reference herein in its entirety.

### Customized Fabrication of Structures Utilizing the FRESH Process

The FRESH process (and, in other implementations, other FRE processes) can be utilized to fabricate an array of different types of medical devices, including synthetic biological structures, artificial grafts, and so on. In one implementation, the FRESH process can be utilized to fabricate functional, biocompatible, hydrogel-based, synthetic biological structures that are customized for the patient's anatomy, such as heart valves, tracheas, skeletal muscle, heart muscle, eye tissue (e.g., the cornea, sclera, anterior chamber, or posterior chamber), bone, cartilage, adipose tissue, neural tissue, and a wide range of other structures for orthopedic, craniofacial, musculoskeletal, cardiovascular, and cosmetic and reconstructive plastic surgery applications. In other implementations, the FRESH process can be utilized to fabricate biocompatible, non-biological structures, such as nerve guidance conduits. While this description focuses on fabricating objects via the FRESH process from hydrogels, the hydrogel FRESH-fabricated objects can also be components within more complex medical devices that additionally incorporate living cells and/or other materials (e.g., non-hydrogel materials). Processes for fabricating these and other example objects will be discussed in greater detail below.

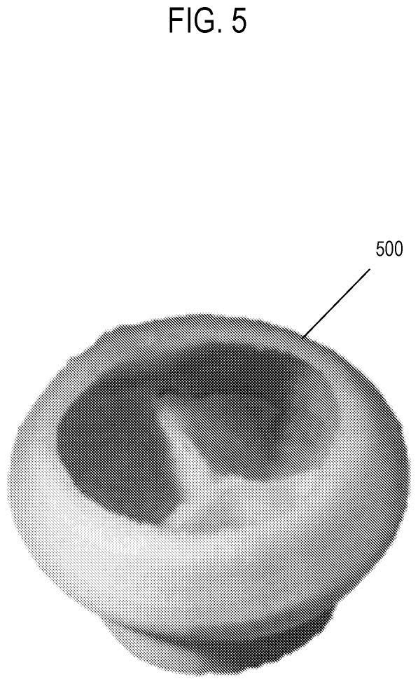

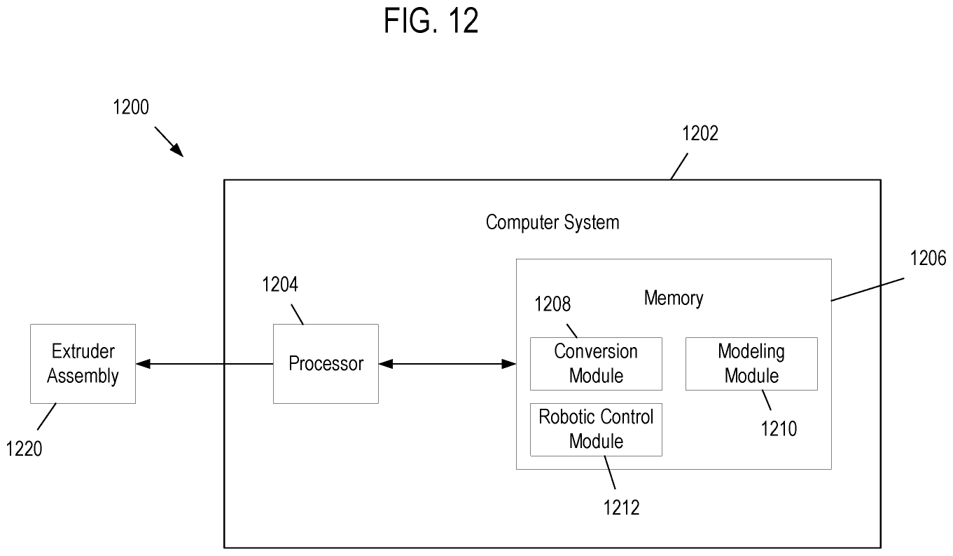

FIG. 4 is a flow diagram of a process 400 for fabricating customized synthetic biological structures. In the following description of the process 400, reference should also be made to FIG. 12, which is a block diagram of an AM system 1200. The process 400 can be implemented in whole or in part as computer-executable instructions stored in a memory 1206 of a computer system 1202 that, when executed by a processor 1204 of the computer system 1202, cause the computer system 1202 to perform the enumerated steps. The computer instructions can be implemented as one or more software modules stored in the memory 1206 that are each programmed to cause the processor 1204 to execute one or more discrete steps of the processes described herein or other functions. In the implementation illustrated in FIG. 12, the computer system 1202 includes a conversion module 1208 programmed to convert the computer model into computer instructions (e.g., G-code) for controlling the movement of the extruder assembly 1220 to fabricate the object defined by the computer model; a modeling module 1210 programmed to receive, store, create, and/or modify computer models of objects to be fabricated; and a robotic control module 1212 programmed to control the movement of the extruder assembly 1220 according to the instructions generated by the conversion module 1208 to fabricate the object. In one aspect, the conversion module 1208 can include slicing software programmed to convert the computer model into a set of planar slices or layers that are to be successively deposited by the extruder assembly 1220 to fabricate the object. In another aspect, the conversion module 1208 can be programmed to convert the computer model into a set of non-planar paths or trajectories for controlling the extruder assembly 1220 to produce 3D filaments, rather than planar layers. Various other modules can be implemented in addition to or in lieu of the aforementioned modules. In another implementation, the processes described herein can be executed across multiple computer systems that are communicably connected together in a network, a computer system communicably connected to a cloud computing system configured to execute one or more of the described steps, and so on.

At a first step 402, the computer system 1202 receives a computer model of a medical device to be fabricated, such as biological structure (e.g., a heart valve, a trachea, or a femur) or an artificial graft (e.g., a nerve guidance conduit). The computer model can be represented in a variety of different formats, such as an STL file or another CAD file format type. In one aspect, the computer model can be constructed from an image of the patient's biological structure that is being replaced or can otherwise be tailored to the patient's anatomy (e.g., by modifying a stock or default computer model to conform to the patient). The image of the patient's biological structure can be obtained via 3D CT scanning, 3D magnetic resonance imaging (MRI), and other such imaging techniques. By utilizing a computer model of the biological structure that is derived directly from the patient, the process 400 described herein can be utilized to fabricate biological structures that are specifically tailored to each individual patient, reducing the failure rate of the replacement structures caused by incompatibility between the geometry of the fabricated structure and the patient's anatomy.

At a second step 404, the computer system 1202 converts the computer model into instructions for controlling the extruder assembly described above or another AM system to fabricate the modeled structure. In various aspects, this can include slicing the computer model into a number of layers of a given thickness and then converting the sliced layers into robotic control instructions (e.g., by a conversion module 1208).

At a third step 406, the computer system 1202 fabricates the structure via a FRE process, such as the FRESH process described above under the heading FRESH PROCESS. Because the FRESH-fabricated object can be based upon a computer model representing the precise biological structure belonging to the patient that is being replaced or augmented, the process 400 can fabricate customized, patient-tailored biological structures.

In other implementations, the process 400 can, at the first step 402, receive a computer model of a medical device that is not a biological structure, such as a nerve guidance conduit. Various examples of biological and non-biological structures that can be fabricated utilizing the process 400 are described below.

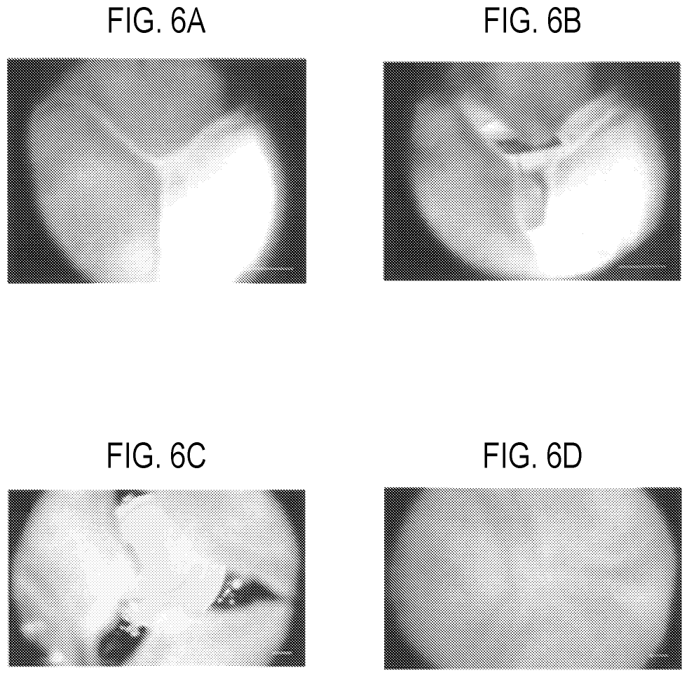

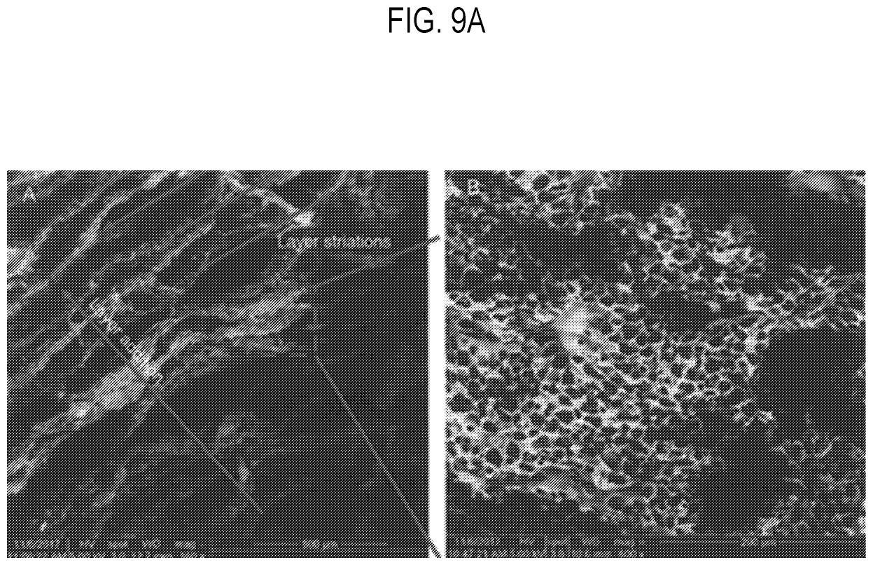

As one example, the process 400 can be utilized to fabricate a heart valve, such as a tricuspid heart valve 500 pictured in FIG. 5. The tricuspid heart valve 500 can be fabricated from a computer model of a natural tricuspid heart valve (or a computer model of a tricuspid heart valve that has been modified to conform to a patient's own tricuspid heart valve), such as the computer model 800 illustrated in FIG. 8A, utilizing the process 400 illustrated in FIG. 4. In one implementation, the tricuspid heart valve 500 can be fabricated utilizing alginate as the structure material and a gelatin support material washed with 0.10% (w/v) CaCl₂. In another implementation, the tricuspid heart valve 500 can be fabricated utilizing collagen as the structure material with a gelatin support material including 50 mM HEPES buffered to pH 7.4. In either implementation, the structure materials can be deposited in the support material in the form of the tricuspid heart valve 500 according to the provided computer model 800 (as described above), heated up to 37° C. for one hour, removed from the liquefied support material, and then transferred to cross-linking solutions (or processed utilizing other cross-linking techniques). During testing, tricuspid heart valves 500 fabricated from alginate were further cross-linked in a 1% (w/v) CaCl₂ solution for one to seven days. Conversely, tricuspid heart valves 500 fabricated from collagen were further washed of all remaining gelatin by placing them in a 1×PBS solution in a rotary incubator at 40° C. and 60 RPM overnight. Afterwards, the fabricated tricuspid heart valves 500 were transferred into a solution of 1×PBS, 0.5% (v/v) glutaraldehyde solution to cross-link for 24 hours. Collagen valves were then placed in 75% (v/v) ethanol, 0.5% (v/v) glutaraldehyde buffered to a pH of 7.4 with 25 mM HEPES for six days to continue cross-linking and prevent infection.

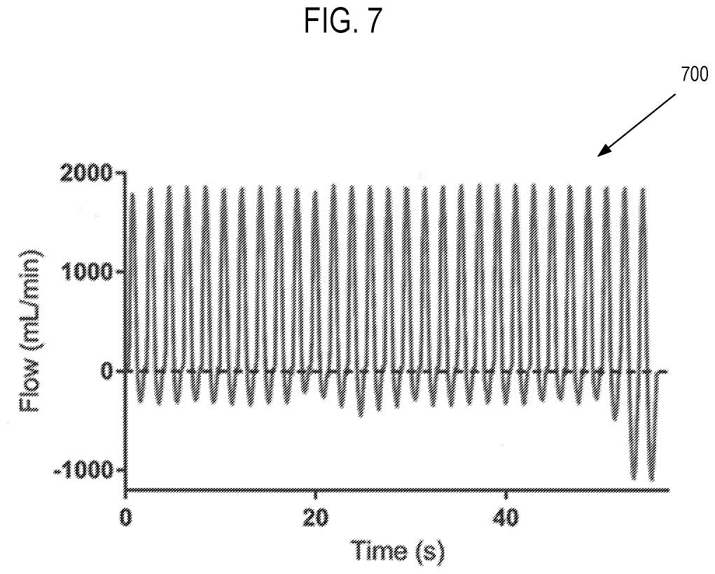

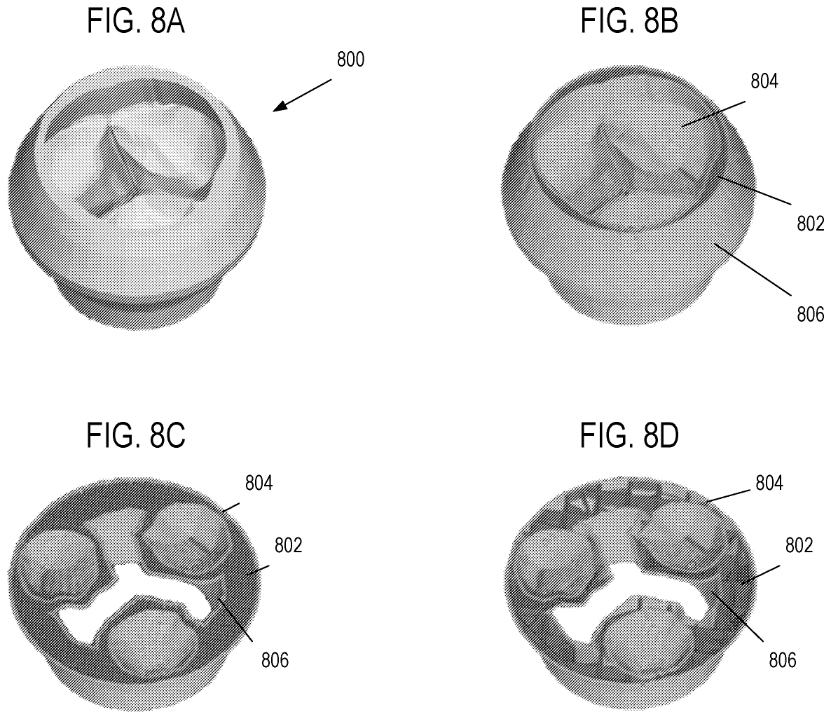

The functionality of the fabricated alginate and collagen tricuspid heart valves 500 were assessed by placing them in a flow loop using a pulsatile pump. FIGS. 6A and 6B are images of an alginate tricuspid heart valve opening and closing under pulsatile flow. FIGS. 6C and 6D are images of a collagen tricuspid heart valve opening and closing under pulsatile flow. This was achieved by replacing the mechanical ball valve on the outlet of the pulsatile pump with the fabricated tricuspid heart valves. Further, an ultrasonic Doppler flow sensor was placed proximal to the valve to assess valvular regurgitation, and a Penrose drain was placed distal to the valve to provide compliance to the system. Still further, a solution of 40% (v/v) glycerol was used as a blood analogue. To mimic the lower transvalvular pressures in the right side of the heart, the pulsatile pump was set to a stroke volume of 30 CC, 30 BPM with a systole time of 33%, resulting in pressures of approximately 25/15 mmHg when using mechanical ball valves. As noted above, the outlet valve was then replaced with a fabricated tricuspid heart valve 500 and then pumped until failure using these same settings. Through Doppler flow velocimetry, it was confirmed that a tricuspid heart valve 500 fabricated utilizing the processes described herein is capable of producing unidirectional flow with regurgitation well below what is deemed to be failure (40% regurgitation). For example, FIG. 7 is a graph 700 of Doppler flow velocimetry across a collagen valve, where the vertical axis represents the flow rate in mL/min and the horizontal axis represents the number of times that the fluid has been pumped through the fabricated tricuspid heart valve 500. The average regurgitation of the fabricated tricuspid heart valve 500 prior to failure was approximately 13%. As can be seen, the FRESH process is capable of producing a tricuspid heart valve 500 that produces sufficient unidirectional flow to be deemed functional.

As another example, the process 400 can be utilized to fabricate a trachea. A trachea consists of a tubular structure that includes a series of C-shaped cartilaginous segments along its length that are more rigid than the remaining portions of the tubular structure. Therefore, a fabricated replacement tracheal structure must likewise include corresponding regions of different rigidities to mimic the biomechanical properties of a natural trachea. In one implementation, the replacement tracheal structure can be fabricated by separately fabricating each of the different tubular and rigid C-shaped components and then joining the separately fabricated tracheal components together to form the complete replacement tracheal structure. The individual tracheal components can be obtained by, for example, segmenting a computer model of the trachea (or portion thereof) being replaced or generating a computer model of each of the components making up the trachea being replaced. The tracheal components can be fabricated utilizing appropriate structure materials and amounts of cross-linking to achieve the desired mechanical properties for each individual tracheal component. In another implementation, the replacement tracheal structure can be fabricated as a singular structure from a computer model of the trachea (or portion thereof) being replaced. The various regions of the replacement tracheal structure can then be selectively cross-linked utilizing various cross-linking techniques, such as the techniques discussed below under the heading FABRICATION TECHNIQUES FOR CONTROLLING MECHANICAL PROPERTIES OF STRUCTURES, to achieve the desired mechanical properties for the different regions of the replacement tracheal structure.

As another example, the process 400 can be utilized to fabricate a medical device that is not a biological structure, such as a nerve guidance conduit. A nerve guidance conduit is an artificial structure for guiding axonal regrowth to facilitate nerve regeneration. In one implementation, a nerve guidance conduit can be printed from a computer model, in the manner discussed above.

In one aspect, various growth agents can be applied to FRESH-fabricated objects to stimulate cell growth or biointegration of the objects. The growth agents can include neurogenesis-inducing agents, angiogenesis-inducing agents, myogenesis-inducing agents, osteogenesis-inducing agents, chondrogenesis-inducing agents, and other growth agents. As one example, various growth agents and other treatments can be applied to a nerve guidance conduit to encourage axonal growth therethrough. Further, the growth agents can be applied to the FRESH-fabricated objects non-uniformly. For example, an axonal growth agent can be applied in a gradient along the length of the nerve guidance conduit such that a higher concentration of the growth agent is present at or near a midpoint of the conduit in order to encourage the axons of neurons positioned at opposing ends of the conduit to growth through the conduit and connect to each other in order to regenerate nerve connections.

The above examples are intended solely to be illustrative of the various concepts described herein. A wide range of other medical devices, including biological and non-biological structures, can be fabricated according to the FRESH process and the techniques described herein.

### Fabrication Techniques for Controlling Mechanical Properties of Structures

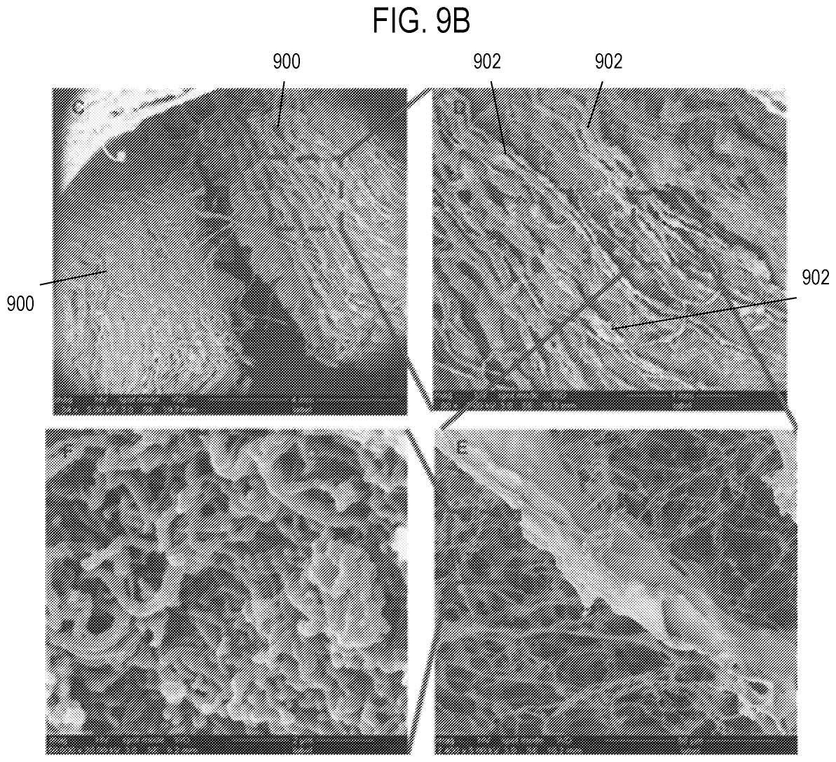

The mechanical properties of objects fabricated utilizing the AM techniques described above can be adjusted by controlling various operational parameters of the FRESH process and/or applying various additional fabrication or post-fabrication techniques to the fabricated objects. In particular, an object's structure can be controlled on several different size scales. At the largest size scales (e.g., hundreds of μm and larger), AM settings such as layer height and infill percentage can control the macroscopic porosity and density of an object. Further, controlling the size of the extruder nozzle can dictate the minimum achievable size (e.g., sub-mm) of features within an object. Still further, evacuating the support material microparticles after fabricating the object can produce a highly porous structure, as can be seen in FIG. 9A. At the below-μm length scales, the structure of the object can be finely controlled by manipulating the chemistry between the support material and the structure material to control the manner in which the polymers of the structure material self-assemble. By manipulating chemistry, hardware, and software choices, the FRESH process allows for the control over sub-micron to macroscopically sized features within an object. Accordingly, each object fabricated via the FRESH process can be customized for a specific application by tailoring its mechanical properties to that application.

For example, objects' mechanical properties can be customized by controlling the extruder assembly 1220 (FIG. 12) to produce more complex movement patterns during the fabrication of the objects. The vast majority of conventional AM is performed using 2D, planar movement, i.e., cutting an object into many slices in the XY or horizontal plane with a chosen thickness in the Z or vertical direction. The extruder nozzle is then moved two-dimensionally through the XY plane to deposit the structure material in striations that are stacked on top of one another in an additive manner to form the object. However, AM systems 1200 (FIG. 12) need not be strictly limited to such 2D movement patterns. In other implementations, the extruder assembly 1220 can be controlled to move in a non-2D manner. In other words, the extruder assembly 1220 can be controlled to move the extruder nozzle three-dimensionally when depositing material, i.e., simultaneously in the X, Y, and Z directions. Further, the extruder assembly 1220 (including the extruder nozzle and/or the platform on which the object is being fabricated) can be rotatable. In such aspects, the instructions for controlling the extruder assembly 1220 for fabricating objects can be defined according to both Cartesian and rotational coordinates, which can allow for the production of objects having complex geometries or very specific mechanical properties. 3D movement during deposition of the structure material allows for an AM system 1200 to build a helical spring in one constant motion, for example. However, even more complex geometries are achievable with robotic arm assemblies capable of simultaneously controlling movement with six degrees of freedom (i.e., in any Cartesian or rotational direction). Accordingly, the process 400 illustrated in FIG. 4 can, at the third step 406, include controlling a rotational orientation or 3D movement of the extruder assembly 1220 during deposition of the structure material.

As another example, objects' mechanical properties can be customized by controlling the infill density or pattern of the fabricated structures. Infill is a repetitive geometric pattern having a defined porosity that is utilized to occupy what would otherwise be empty spaces within an additively manufactured object. As illustrated in FIGS. 8C and 8D, which are sectional views of a computer model 800 of a heart valve (FIG. 8A) after it has been processed by slicing software (FIG. 8B), infill 802 is located between an outer wall 804 and an inner wall 806 of the heart valve structure 800. Infill density can be represented, for example, as a percentage from 0-100%, where 0% represents a complete hollow space and 100% represents a solid object. To illustrate how infill density can be controlled to adjust the varying degrees of solidity of the heart valve structure 800, FIG. 8C illustrates a computer model of a heart valve structure 800 having an infill density of 50% and FIG. 8D illustrates a computer model of the same heart valve structure 800, except where the infill density is 10%. Infill density can affect the weight, strength, and other mechanical properties of the structure. Furthermore, infill can be fabricated in a variety of different patterns, such as grids, lines, honeycomb structures, and so on. Various infill patterns can be more suitable for differently shaped structures and/or change the mechanical properties of the structure (e.g., provide non-uniform strength characteristics). Still further, structures (or components thereof) can be fabricated to have non-uniform infill densities and/or patterns throughout the structure. Therefore, different portions or components of the fabricated structures can have different mechanical properties. Accordingly, the process 400 illustrated in FIG. 4 can, at the third step 406, include controlling a density or pattern of the infill 802 of the FRESH-fabricated object.

As another example, objects' mechanical properties can be customized by controlling the directions or patterns in which the structure material is deposited. During fabrication of the object, the structure material can be deposited by the extruder assembly 1220 as a series of successive planar or arbitrary 3D striations that fuse together to ultimately form the object. As can be see in FIG. 9A, the longitudinal axes of the striations are orthogonal to the direction in which the layers or striations are added. The striations 902 are anisotropic, exhibiting different mechanical properties (e.g., tensile strength) along their longitudinal axes than their lateral axes, which in turn affects the mechanical properties of the object. Therefore, controlling the direction in which the striations 902 are deposited to form the object allows one to control the mechanical properties of the object. Further, as noted above, the directions in which the striations are deposited can be in arbitrary 3D space and are not limited to planar movements. For example, if it was desired for the object to exhibit a higher tensile strength in a particular direction, the extruder assembly 1220 could be controlled to deposit the structure material such that the longitudinal axes of the striations were aligned with that desired direction. Further, the direction in which it is desired to deposit a material can be determined through imaging of the biological structure. In one implementation, the described techniques for controlling the movement of the extruder assembly 1220 in a non-planar manner can be utilized to fabricate an object that mimics the mechanical properties of the corresponding biological structure. For example, the 3D orientation of the muscle fibers within the wall of the heart affect the mechanical and functional properties of the heart. Accordingly, the heart can be imaged utilizing an imaging technique (e.g., diffusion tensor imaging) to determine the 3D orientation of the muscle fibers in the wall of the heart. Once the overall structure of the heart and the orientation of the muscles fibers is determined and a computer model including this information is generated, the computer model can be converted (e.g., by a conversion module 1208) into computer instructions for controlling the extruder assembly 1220 to fabricate the imaged heart from FRESH-printed hydrogel and/or cells with fibers or striations arranged in the same complex, 3D manner as the imaged muscle fibers. For example, FIG. 9B shows a series of increasingly higher magnification images showing two leaflets 900 of a heart valve fabricated from collagen in which the deposited striations 902 are visible. The directions in which the striations 902 of the structure material are arranged can thus correspond to the directions in which the muscle fibers of the natural heart valves are arranged in order to mimic the properties of those muscle fibers. Accordingly, the process 400 illustrated in FIG. 4 can, at the third step 406, include controlling the extruder assembly 1220 such that the striations (or a portion of the striations) were aligned with a particular direction in which it was desired for the FRESH-fabricated object to exhibit particular properties (e.g., increased tensile strength). Further, by imaging the biological structure and controlling the direction and/or orientation of the deposited structure material based upon the structural properties of the biological structure (e.g., muscle fiber direction), one can create a medical device and/or tissue having the same anisotropic mechanical, electrical, and/or structural properties of the imaged biological structure in order to recreate normal tissue/organ function

As another example, objects' mechanical properties can be customized by controlling the amount of cross-linking applied to the fabricated object and/or the locations at which the fabricated object is cross-linked utilizing photo, ionic, enzymatic, or pH/thermally driven mechanisms. As discussed above under the heading FRESH PROCESS, cross-linking can be utilized to increase the rigidity of the deposited structure material. In an implementation for chemical-induced and similar cross-linking mechanisms, one can control the areas of the object that are exposed to the cross-linking chemical(s). For example, when fabricating a trachea replacement structure, the cross-linking chemicals(s) can be selectively applied to the regions of the object corresponding to the tracheal rings, thus causing those regions to be selectively stiffer than the remaining regions of the object. In an implementation for photo-induced cross-linking, one can control the areas of the object that are exposed to the UV light by selectively covering the areas where it is desired to avoid or minimize cross-linking of the structure material. For example, when fabricating a trachea replacement structure, the regions of the object corresponding to the tracheal rings can be exposed to (and the remaining regions can be covered from) the UV light to selectively induce cross-linking of the structure material at those locations. Accordingly, the process 400 illustrated in FIG. 4 can, at the third step 406, include selectively cross-linking a portion of the FRESH-fabricated object.

### Gauging

One issue with any manufactured product, including additively manufactured biological structures or other medical devices, is ensuring that the manufactured product conforms to the required dimensions and mechanical constraints. Because the additively manufactured medical devices described herein begin as a computer model that is converted to machine movement control instructions to fabricate the physical object, one can gauge or assess the quality of the fabricated objects by comparing 3D images of the fabricated objects to the source computer model. Accordingly, the 3D dimensions of the fabricated objects can be compared to the computer model to assess how accurately the object was fabricated. Precisely capturing an object's dimensions is possible through a variety of techniques, including both imaging techniques (e.g., CT, MRI, optical coherence tomography (OCT), laser scanning, or ultrasound) and non-imaging techniques (e.g., probing). Once obtained, the 3D image or reconstruction of the fabricated object can then be compared to the source computer model to determine the dimensional accuracy of the fabricated objected. Some of these gauging techniques have been utilized in the context of gauging machine parts (e.g., metal turbine blades); however, they have not been utilized in the context of gauging additively printed soft hydrogel structures, as is described herein.

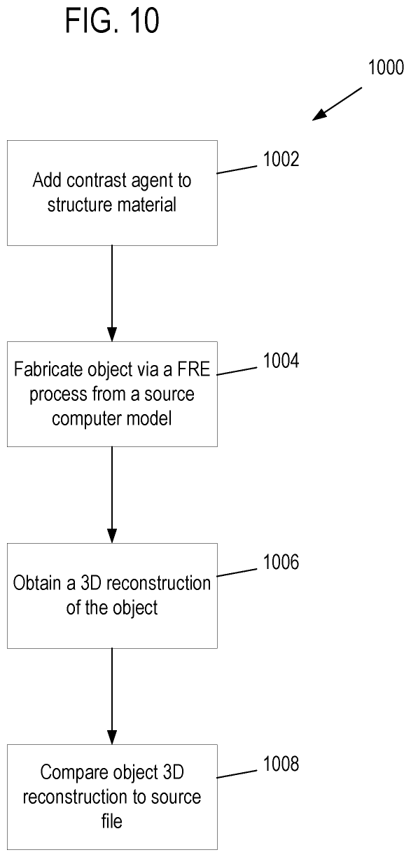

FIG. 10 is a flow diagram of one implementation of a process 1000 for gauging a fabricated structure, such as a replacement biological structure fabricated via the FRESH process. At a first step 1002, a contrast agent is added to the structure material prior to manufacturing the replacement biological structure. The contrast agent can be selected based upon the imaging technique that is to be utilized to image the fabricated object. For example, the contrast agent can include a radiocontrast agent (e.g., barium sulfate) if CT (contrast CT), projection radiography, or other such imaging techniques are going to be utilized to image the fabricated object. As another example, the contrast agent can include a MRI contrast agent (e.g., a gadolinium(III)-based contrast agent) if MRI is going to be utilized to image the fabricated object. Various other contrast agents can be utilized for other imaging techniques.

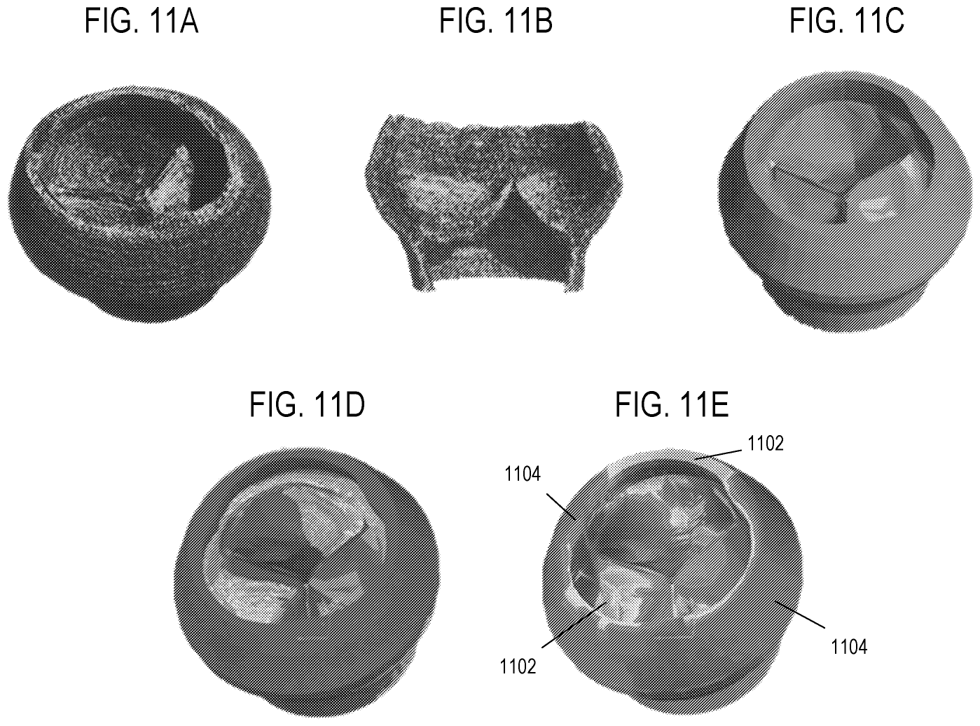

At a second step 1004, the object (e.g., a replacement biological structure or a non-biological structure, such as a nerve guidance conduit) is fabricated utilizing the FRESH process, as described above. At a third step 1006, a 3D reconstruction of the fabricated object is obtained via CT, MRI, OCT, or other imaging techniques. The contrast agent present in the structure material from which the replacement structure was fabricated improves the ability for the fabricated object to be imaged clearly. For example, FIGS. 11A and 11B illustrate a 3D reconstruction captured via a CT scan of a tricuspid heart valve fabricated utilizing the FRESH process.

At a fourth step 1008, the 3D reconstruction of the object is compared to the source computer model from which the object was fabricated. In one aspect, the 3D reconstruction and the computer model can be compared by overlaying them and then performing a surface deviation analysis to determine whether and where the replacement structure was over- or underprinted. For example, FIG. 11C illustrates the computer model from which the object shown in FIGS. 11A and 11B was fabricated. Further, FIG. 11D illustrates an overlay of the 3D reconstruction and the computer model, and FIG. 11E illustrates a surface deviation analysis of the overlay shown in FIG. 11D. As can be seen in FIG. 11E, the deviation analysis can indicate both overprinted regions 1102 (i.e., regions where the object surface exceeds the dimensional boundaries delineated by the computer model), which are indicated by the lighter shades, and underprinted regions 1104 (i.e., regions where the object surface is below the dimensional boundaries delineated by the computer model), which are indicated by the darker shades.

This process 1000 can be utilized to ensure that FRESH-fabricated objects, such as replacement biological structures, that are customized for individual patients are in fact within established mechanical and anatomical tolerances for that patient. If a fabricated object is outside of the established tolerances, then the object can undergo post-fabrication processing (e.g., shaving or reshaping) to correct any issues, Alternatively, the defective object can be discarded and a new object can be fabricated. This can prevent any issues that can arise from surgically fitting a patient with a FRESH-fabricated object that is not a complete anatomical match with the patient or that has any structural or mechanical irregularities.

### Surgical Techniques

The processes for surgically fitting a patient with a replacement biological structure can vary greatly in terms of invasiveness and complexity based upon the particular biological structure being replaced or augmented. For example, open-heart surgical procedures to repair or replace heart valves are incredibly invasive procedures. Although less invasive than open-heart surgical procedures, even nominally minimally invasive heart valve repair or replacement procedures are still relatively invasive and require multiple incisions and manipulation by multiple surgical instruments inserted into the patient's chest cavity. Therefore, minimally invasive surgical techniques to deliver biological replacement structures fabricated utilizing the FRESH process are desirable. In one implementation, a replacement biological structure, such as a replacement heart valve, can be fabricated such that it has a low axial rigidity or otherwise can be folded or compressed into a size that is translatable through a vascular pathway. Accordingly, the compressed replacement biological structure can be affixed to a balloon catheter and then delivered to the appropriate location within the body through the vascular pathway. Once at the appropriate location, the balloon can be inflated to unfold or decompress the replacement biological structure into its operational shape. For example, a replacement heart valve can be fabricated utilizing the FRESH process, delivered to the location of the patient's heart valve being replaced or supplemented by the replacement heart valve, and then deployed throughout inflation of the balloon.

### Examples

Various aspects of the subject matter described herein are set out in the following aspects, implementations, and/or examples, which can be interchangeably combined together in various arrangements:

In one general aspect, a method of fabricating a replacement structure for a biological structure of a patient. The method comprising: (i) depositing a structure material into a support material in the form of the replacement structure based upon a computer model generated from image data of the biological structure of the patient; wherein the support material is stationary at an applied stress level below a threshold shear stress level and flows at an applied shear stress level at or above the threshold shear stress level; wherein the support material is configured to physically support the structure material during deposition of the structure material; wherein the structure material comprises a fluid that transitions to a solid or semi-solid state after deposition; (ii) removing the support material; and (iii) inducing cross-linking of the structure material of the replacement structure.

In one aspect, the method further comprises: obtaining the image data of the biological structure from the patient; and generating the computer model of the biological structure from the image data of the biological structure.

In one aspect, obtaining the image data of the biological structure comprises scanning a patient with a CT scan.

In one aspect, obtaining the image data of the biological structure comprises scanning a patient with an MRI scan.

In one aspect, obtaining the image data of the biological structure comprises scanning a patient with an OCT scan.

In one aspect, obtaining the image data of the biological structure comprises scanning a patient with a laser scan.

In one aspect, obtaining the image data of the biological structure comprises scanning a patient with an ultrasound scan.

In one aspect, the replacement structure is selected from the group consisting of a heart valve and a trachea.

In one aspect, the structure material comprises a hydrogel comprising a material selected from the group consisting of collagen, alginate, decellularized extracellular matrix material, fibrinogen, Matrigel, and hyaluronic acid.

In one aspect, the support material comprises a hydrogel comprising a gelatin microparticle slurry.

In one aspect, the method further comprises applying a growth agent to the replacement structure.

In one aspect, the growth agent is selected from the group consisting of a neurogenesis-inducing agent, an angiogenesis-inducing agent, a myogenesis-inducing agent, an osteogenesis-inducing agent, and a chondrogenesis-inducing agent.

In one aspect, treating the replacement structure comprises treating a selected portion of the replacement structure to create a differential rigidity in the replacement structure.

In one aspect, wherein the structure material comprises a contrast agent, the method further comprises: imaging the replacement structure according to the contrast agent; and comparing the image of the replacement structure with the computer model.

In one aspect, imaging the replacement structure comprises capturing the image of the replacement structure via an imaging technique, the imaging technique selected from the group consisting of CT, MRI, OCT, laser scanning, and ultrasound.

In one aspect, the method further comprises surgically fitting the replacement structure in a patient from whom the image data of the biological structure was captured.

In one aspect, the support material comprises a thermoreversible material and removing the support material comprises heating the support material to a threshold temperature at which the support material transitions from a solid or semi-solid state to a liquid state.

In one aspect, depositing the structure material into the support material comprises depositing the structure material such that a longitudinal axis of a striation of the deposited structure material is aligned with a predetermined direction to cause the replacement structure to exhibit anisotropic properties.

In one aspect, depositing the structure material into the support material comprises depositing the structure material in a non-planar direction to cause the replacement structure to exhibit anisotropic properties.

In one aspect, the method further comprises: obtaining the image data of the biological structure from the patient; and determining a direction of a fiber of the biological structure; wherein depositing the structure material into the support material comprises depositing the structure material in a direction aligned with the direction of the fiber of the biological structure.

In one aspect, the biological structure comprises a heart and the fiber comprises a muscle fiber.

In one aspect, inducing cross-linking of the structure material of the replacement structure comprises selectively treating a portion of the replacement structure with the cross-linking agent such that cross-linking of the structure material is induced in that portion.

In another general aspect, a patient-customized, embedded- and additive-printed hydrogel material in the form of a body part for a patient, wherein the hydrogel material comprises cross-linked polymers, fabricated according to any of the methods described above.

The examples presented herein are intended to illustrate potential and specific implementations of the present invention. It can be appreciated that the examples are intended primarily for purposes of illustration of the invention for those skilled in the art. No particular aspect or aspects of the examples are necessarily intended to limit the scope of the present invention. Further, it is to be understood that the figures and descriptions of the present invention have been simplified to illustrate elements that are relevant for a clear understanding of the present invention, while eliminating, for purposes of clarity, other elements. While various embodiments have been described herein, it should be apparent that various modifications, alterations, and adaptations to those embodiments may occur to persons skilled in the art with attainment of at least some of the advantages. The disclosed embodiments are therefore intended to include all such modifications, alterations, and adaptations without departing from the scope of the embodiments as set forth herein.

1. A method of fabricating a replacement structure for a biological structure of a patient, the method comprising:
depositing a structure material into a support material in the form of the replacement structure based upon a computer model generated from image data of the biological structure of the patient;
wherein the support material is stationary at an applied stress level below a threshold shear stress level and flows at an applied shear stress level at or above the threshold shear stress level;
wherein the support material is configured to physically support the structure material during deposition of the structure material;
wherein the structure material comprises a fluid that transitions to a solid or semi-solid state after deposition;
wherein the structure material comprises a contrast agent;
removing the support material; and
inducing cross-linking of the structure material of the replacement structure;
imaging the replacement structure according to the contrast agent; and
comparing the image of the replacement structure with the computer model.

2. The method of claim 1, further comprising:
obtaining the image data of the biological structure from the patient; and
generating the computer model of the biological structure from the image data of the biological structure.

3. The method of claim 2, wherein obtaining the image data of the biological structure comprises scanning a patient with a CT scan.

4. The method of claim 2, wherein obtaining the image data of the biological structure comprises scanning a patient with an MRI scan.

5. The method of claim 2, wherein obtaining the image data of the biological structure comprises scanning a patient with an OCT scan.

6. The method of claim 2, wherein obtaining the image data of the biological structure comprises scanning a patient with a laser scan.

7. The method of claim 2, wherein obtaining the image data of the biological structure comprises scanning a patient with an ultrasound scan.

8. The method of claim 1, wherein the replacement structure is selected from the group consisting of a heart valve and a trachea.

9. The method of claim 1, wherein the structure material comprises a hydrogel comprising a material selected from the group consisting of collagen, alginate, decellularized extracellular matrix material, fibrinogen, Matrigel, and hyaluronic acid.

10. The method of claim 1, wherein the support material comprises a hydrogel comprising a gelatin microparticle slurry.

11. The method of claim 1, further comprising applying a growth agent to the replacement structure.

12. The method of claim 11, wherein the growth agent is selected from the group consisting of a neurogenesis-inducing agent, an angiogenesis-inducing agent, a myogenesis-inducing agent, an osteogenesis-inducing agent, and a chondrogenesis-inducing agent.

13. The method of claim 1, wherein treating the replacement structure comprises treating a selected portion of the replacement structure to create a differential rigidity in the replacement structure.

14. (canceled)

15. The method of claim 1, wherein imaging the replacement structure comprises capturing the image of the replacement structure via an imaging technique, the imaging technique selected from the group consisting of CT, MRI, OCT, laser scanning, and ultrasound.

16. The method of claim 1, further comprising surgically fitting the replacement structure in a patient from whom the image data of the biological structure was captured.

17. The method of claim 1, wherein:
the support material comprises a thermoreversible material; and
removing the support material comprises heating the support material to a threshold temperature at which the support material transitions from a solid or semi-solid state to a liquid state.

18. The method of claim 1, wherein depositing the structure material into the support material comprises depositing the structure material such that a longitudinal axis of a striation of the deposited structure material is aligned with a predetermined direction to cause the replacement structure to exhibit anisotropic properties.

19. The method of claim 1, wherein depositing the structure material into the support material comprises depositing the structure material in a non-planar direction to cause the replacement structure to exhibit anisotropic properties.

20. The method of claim 1, further comprising:
obtaining the image data of the biological structure from the patient; and
determining a direction of a fiber of the biological structure;
wherein depositing the structure material into the support material comprises depositing the structure material in a direction aligned with the direction of the fiber of the biological structure.

21. The method of claim 20, wherein the biological structure comprises a heart and the fiber comprises a muscle fiber.

22. The method of claim 1, wherein inducing cross-linking of the structure material of the replacement structure comprises selectively treating a portion of the replacement structure with the cross-linking agent such that cross-linking of the structure material is induced in that portion.

23. A product fabricated by the method of claim 1.

24-36. (canceled)
